(19) United States
(12) Patent Application Publication
(10) Pub. No.: US 2003/0175314 A1
(43) Pub. Date: Sep. 18, 2003

(54) Pharmaceutical composition for dermal application

(76) Inventor: Erik Johannes Didriksen, Ballerup (DK)

Correspondence Address: BIRCH STEWART KOLASCH & BIRCH, PO BOX 747, FALLS CHURCH, VA, 22040-0747, US

(21) Appl. No.: 10/295,945

(22) Filed: Nov. 18, 2002

Related U.S. Application Data
(60) Provisional application No. 60/331,539, filed on Nov. 19, 2001.

(51) Int. Cl. A61K 9/00

(52) U.S. Cl. 424/400

(57) ABSTRACT

A pharmaceutical composition for dermal application comprising, as an active component, a

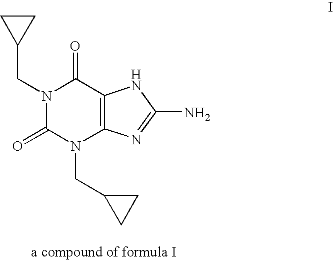

or a pharmaceutically acceptable salt thereof, and a vehicle comprising a pharmaceutically acceptable, substantially non-aqueous solvent in which the active component has a solubility of at least about 6.0 g/L at ambient temperature, said solvent being capable of effecting penetration of a therapeutically effective amount of the active component into the epidermis, may be used in the prevention or treatment of dermal inflammatory diseases or conditions such as dermatitis.

### FIELD OF INVENTION

The present invention relates to a pharmaceutical composition for dermal application as well as the use of the composition in the prophylaxis and treatment of dermal conditions.

### BACKGROUND OF THE INVENTION

Dermal diseases affect a large number of patients with a prevalence (in the UK) of about 20%. Dermal inflammatory diseases are the most common dermal diseases, constituting about 50% of the skin diseases seen in general practice. Skin diseases may have profound psychosocial consequences, e.g. for patients suffering from extensive psoriasis or eczema. Dermal inflammatory conditions include dermatitis (eczema) typically involving redness or swelling of the skin or lesion which may be oozing, scaling or crusting. The inflamed areas of the skin are often also itchy (pruritic). Examples of dermatitis are atopic dermatitis, contact dermatitis or seborrheic dermatitis.

Atopic dermatitis is a chronic, relapsing, pruritic inflammatory skin disease occurring in genetically predisposed individuals. The onset of the disease is mostly in infancy or early childhood, 80% of the cases occurring before the age of 1 year. While there is a general tendency towards spontaneous improvement throughout childhood, approximately 60% of patients with severe atopic dermatitis and approximately 40% of patients with moderate atopic dermatitis contend with skin problems as adults, most frequently hand eczema.

The clinical manifestations of atopic dermatitis are erythema (redness), vesiculopapules, oozing or crusting, lichenification and general dryness of the skin. As the main complaint of atopic dermatitis is pruritus, excoriations are also prevalent.

Even though the disease has been under intense investigation with respect to pathogenesis and genetics in recent years, no pathognomic biochemical marker has been identified so far. There is general agreement that atopic dermatitis is a complex, multifactorial disease in which both genetic and environmental factors are involved. The role of allergens in the development of atopic dermatitis is controversial, and it has not been possible to demonstrate a consistent link between allergens and the disease.

Atopic disease is regarded as a condition characterised by hyper-reactivity both at the clinical and the cellular level. One theory is that an unknown genetic predisposition leads to an imbalance of the immune system favouring the differentiation of T-helper-2 (Th2)-type immunological characteristics such as enhanced IgE levels, blood eosinophilia and increased numbers of Th2-type cells. The cytokines involved are interleukin (IL)-4, IL-5 and IL-10 inducing secretion of IL-12 from eosinophils and macrophages. The IL-12 leads to activation of Th1 and Th0 cells which are responsible for the production of interferon γ (IFN-γ). The level of IFN-γ is correlated to the clinical severity of atopic dermatitis.

Recent studies indicate that patients suffering from atopic dermatitis have fundamental defects in their blood monocytes. Atopic leukocytes have an abnormally high activity of cyclic adenosine monophosphate phosphodiesterase (PDE) isoenzymes most prominent in monocytes, but an atopic PDE abnormality may also be present in multiple leukocyte subsets including basophils, B cells, T cells and eosinophils.

Current therapies of atopic dermatitis include topical treatment with emollients, topical corticosteroids and tar preparations, systemic treatment with corticosteroids, antibiotics, cyclosporine and antihistamines as well as phototherapy. The mainstay of therapy is topical corticosteroids which involve the risk of adverse cutaneous and systemic effects, including skin atrophy. New topical treatments have been proposed involving the application of ascomycin derivatives with an immunosuppressive effect. The long-term consequences of immunosuppressive treatment of atopic dermatitis have not been established, but some adverse effects including a higher incidence of infection in patients treated with ascomycin may be anticipated. Consequently, there is a need for an effective anti-inflammatory treatment of atopic dermatitis with fewer adverse effects.

Contact dermatitis is an inflammatory condition of the skin which appears when skin is in contact with an irritant or sensitizing substance at a level above the resistance threshold value of an individual. The onset of contact dermatitis may be occasioned by physicochemical properties of the sensitizing/irritant substance, sensitization activity, contact frequency or predisposition of the affected individual, or a combination thereof. The clinical symptoms are very like those of atopic dermatitis, i.e. rash, erythema, edema, appearance of vesiculopapules and pruritis. The mechanism of onset is considered to involve a type IV allergic reaction induced by the reaction of sensitized T-cells with antigen resulting in release of cytokines from the T-cells.

Pruritus is a condition observed in a variety of dermal inflammatory diseases as well as in a number of systemic diseases such as malignant tumours, diabetes mellitus, hepatic diseases, renal failure, hemodialysis, thyroid diseases etc. Patients with pruritus experience a great deal of discomfort, and in severe cases it may cause significant disruption of daily life. In particular in connection with dermatitis, treatment for pruritus is required because scratching of the skin by the patient causes an aggravation of the symptoms.

Excoriation is the most exacerbating factor of dermatitis because scratching injures the skin resulting in defect of the barrier function and erosion by physical or chemical stimuli, and bacterial infection readily occurs. Due to extensive scratching of affected skin, even during sleep, patients affected by atopic dermatitis may develop atopic exanthema.

Because of the negative psychosocial consequences for affected individuals, and the relatively limited numbers of drugs available for topical treatment of dermal inflammatory diseases and the severity of the known side effects of some of these drugs, the provision of new medicaments for adequate therapy of dermal inflammatory diseases is very important.

### SUMMARY OF THE INVENTION

The object of the present invention is to provide a topical formulation for application onto inflamed skin, such application resulting in effective penetration of an active anti-inflammatory component of the formulation into the epidermis while excluding excipient materials in the vehicle which might give rise to exacerbation of the skin irritation typically found in dermal inflammatory conditions.

Accordingly, the invention relates to a substantially non-aqueous pharmaceutical composition for dermal application comprising, as an active component, a compound of formula I

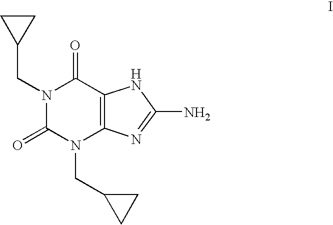

or a pharmaceutically acceptable salt thereof, and a vehicle comprising a pharmaceutically acceptable, substantially non-aqueous solvent in which the active component has a solubility of at least about 6.0 g/L at ambient temperature, said solvent being capable of effecting penetration of a therapeutically effective amount of the active component into the epidermis.

In the present context, the term “substantially non-aqueous” is intended to indicate that the composition has a water content below about 2%, such as below about 1.5%, in particular below about 1%.

In another aspect, the invention relates to the use of said pharmaceutical composition for the manufacture of a medicament for the prophylaxis or treatment of dermal inflammatory diseases or conditions.

In a further aspect, the invention relates to a method of preventing or treating dermal inflammatory diseases or conditions, the method comprising dermally applying to a site of inflammation an effective amount of said pharmaceutical composition.

### DETAILED DESCRIPTION OF THE INVENTION

The compound of formula I, 1,3-dicyclopropylmethyl-8-aminoxanthine, is also known by the proposed generic name cipamfylline. The compound and a method of its preparation is disclosed in U.S. Pat. No. 5,734,051 which is hereby incorporated by reference in its entirety. In in vitro studies, this compound has been shown to be a potent and selective inhibitor of PDE₄ and the secretion of proinflammatory cytokines such as tumour necrosis factor α (TNF-α), IL-10, IL-12 and IFN-γ, but has no effect on lymphocyte proliferation contrary to rapamycin and tacrolimus (both immunosuppressive agents currently in clinical trials for atopic dermatitis).

Cipamfylline has a pronounced inner crystal bonding resulting in a high melting point (about 310-312° C.) and some degree of hydrophobicity. Consequently, cipamfylline is sparingly soluble, generally having a solubility in water of less than 0.05 g/L (0.005%) as well as being slowly soluble (with an intrinsic dissolution rate in the range of from about 0.0064 mg/min/cm² to 0.0096 mg/min/cm²). On the other hand, effective delivery of the active component into the epidermis where it can exert its effect requires that the compound be present in a dissolved state in said composition. In the course of research leading to the present invention, it has been found that certain semipolar alkylene glycols and alkylene glycol ethers are capable of dissolving a therapeutically effective amount of the active component, in particular in the absence of water as a co-solvent. It has been found that if water is present as a co-solvent in an amount of about 25% or more, the solubility of cipamfylline is greatly decreased, which means that a lower quantity of the active component is available for penetration into the skin.

Consequently, the solvent should be one in which the active component has a solubility of at least about 7 g/L, such as at least about 8 g/L, at least about 9 g/L, at least about 10 g/L, at least about 11 g/L, at least about 12 g/L, at least 13 g/L, at least about 14 g/L, at least about 15 g/L, at least about 16 g/L, at least about 17 g/L, or at least 18 g/L, at ambient temperature.

More specifically, the solvent comprises a non-toxic glycol or glycol ether selected from the group consisting of propylene glycol, butylene glycol, diethylene glycol and diethylene glycol ether. Of these solvent substances, propylene glycol has been found to result in a particularly favourable dissolution of the active component. The advantageous dissolution properties of propylene glycol may be explained by the solubility properties of this particular solvent, as determined by the Hildebrand solubility coefficient (J H Hildebrand and R L Scott, The Solubility of Non-Electrolytes, Reinhold, N.Y., 1949). The Hildebrand coefficient (solubility parameter δ) for propylene glycol have been found to be about 14.8 (cal/cm³)^{1/2} and that of cipamfylline has been found to be about 13.4 (cal/cm³)^{1/2}. Thus propylene glycol has a Hildebrand coefficient within ±2 units of that of cipamfylline which makes it an effective solvent for this active component.

Apart from being a solvent for cipamfylline, propylene glycol as a secondary penetration enhancer, i.e. a substance which is capable of penetrating the stratum corneum and “draw” low-molecular components such as therapeutically active components in the vehicle into the epidermis. While in our research propylene glycol does not in itself give rise to any significant skin irritation, it is capable of “drawing” low-molecular and potentially irritative components of the vehicle into the epidermis, leading to an overall irritative effect of conventional vehicles including propylene glycol. For this reason, propylene glycol has previously been thought to be unsuitable for inclusion as a solvent in compositions intended for the treatment of atopic dermatitis. However, the present vehicle has been specifically designed to exclude all excipients which are capable of penetrating into the epidermis (or being “drawn” into the epidermis by the solvent) and which are not essential for formulation purposes, resulting in a composition which, according to the clinical tests described in Example 3 herein, gives rise to fewer irritative reactions in skin on which the composition is applied compared to a conventional reference formulation. This, of course, is particularly important for dermal formulations intended for application on inflamed, and therefore highly irritated skin so as to avoid a painful burning sensation at the site of application and more long-term irritation such as pain or itching.

Substantially non-aqueous formulations suitable for dermal application include liquid or semi-liquid preparations such as ointments or pastes.

The present composition may further comprise an emulsifier. This may be included to adjust the size of the droplets of oil in the oil phase, which may have a diameter of up to 200 μm, to a smaller size, typically in the range of 1-50 μm, thereby improving the cosmetic appearance of the composition. The emulsifier may suitably be a water-in-oil emulsifier, e.g. selected from the group consisting of water-in-oil emulsifiers such as, for example, polyoxyalkylene C₁₂₋₂₀ alkyl ether, such as polyoxyethylene-2-cetyl ether, polyoxyethylene-2-lauryl ether, polyoxyethylene-2-oleyl ether or polyoxyethylene-2-stearyl ether, sorbitan oleate, sorbitan isostearate, sorbitan sesquioleate, glycerol esters of isostearic acid and adipic acid, polyglyceryl-3-diisostearate and polyglyceryl-6-hexaricinoleate.

In a currently favoured embodiment of the present composition, the vehicle comprises

(a) a solvent for the active component in an amount of about 5-20% w/w, e.g. about 10-15% w/w,

(b) a water-in-oil emulsifier in an amount of about 1.5-7.5% w/w, e.g. about 3-5% w/w, and

(c) an oil base as the remainder.

The amount of the individual ingredients in the composition will, to some extent, depend on the concentration of the active component incorporated therein. By way of example, at a concentration of the active component of 2.5 mg/g of vehicle, the amount of propylene glycol solvent required to dissolve this amount of active component is typically in the range of from about 10% w/w to about 15% w/w of the vehicle. At lower concentrations of the active component, the amount of propylene glycol solvent may be correspondingly reduced.

The amount of active component in the composition may vary according to the severity of the condition to be treated, but will generally be in the range of from about 0.5 to about 3.5 mg/g of vehicle, in particular about 1.0-3.0 mg/g of vehicle, such as about 2.5 mg/g of vehicle.

The composition of the present invention may be prepared in accordance with methods well known to the person skilled in the art of pharmaceutical formulation. Thus, the composition may be prepared by incorporating the ingredients into a well known cream or ointment base comprising an oil, preferably an oil which is not soluble in the solvent for the active component and which is therefore not “drawn” into the epidermis, thereby potentially causing skin irritation. Examples of suitable oils include liquid paraffin, white soft paraffin or straight or branched hydrocarbons of, for instance, 14-18 carbon atoms, such as isohexadecane.

In addition to the above-mentioned ingredients, the present composition may include one or more additional ingredients such as other therapeutically active substances applied in the tratment of dermal inflammatory conditions, including corticosteroids such as hydrocortisone, salicylic acid or topical antibiotics such as clindamycin. Furthermore, the present composition may include a topical anesthetic such as bupivacaine, chlorprocaine, dibucaine, ketamine, pramoxine or the like.

The present composition may also comprise other components commonly used in dermal formulations, e.g. antioxidants (e.g. alpha-tocopherol), preservatives, emollients, pigments, skin soothing agents, skin healing agents and skin conditioning agents such as urea, glycerol, allantoin or bisabolol, cf. CTFA Cosmetic Ingredients Handbook, 2^{nd} Ed., 1992.

The dermal inflammatory disease or condition to be treated by the present method or medicament is, in particular, dermatitis (eczema), such as atopic dermatitis, seborrheic dermatitis or contact dermatitis, urticaria or pruritis.

The dosage in which the active component is administered may vary between wide limits, depending on the age and condition of the patient, the severity of the condition to be treated and the discretion of the physician. A suitable dose of the present composition comprising a compound of formula I will, however, generally be in the range of about 1-3 mg/cm² applied on the affected area or areas of the skin one or more times a day.

The present invention is described in further detail in the following examples which are not in any way intended to limit the scope of the invention as claimed.

### EXAMPLES

### Example 1

Preparation of a Dermal Ointment

A. Cipamfylline (2.5 mg/g vehicle) was dissolved in propylene glycol (150 mg/g vehicle). Macrogol-2-stearyl ether (50 mg/g vehicle) (e.g. Brij® 72) was melted with liquid paraffin (50 mg/g vehicle) and white soft paraffin (747.5 mg/g vehicle) heated to 70-80° C. The cipamfylline solution was slowly added to the oil phase and the resulting ointment was thoroughly homogenised and cooled to room temperature with mild agitation. The resulting formulation was filled into 50 g tubes and stored at room temperature.

B. A reference cream formulation was prepared by by dissolving cipamfylline (1.5 mg/g vehicle) in propylene glycol (480 mg/g vehicle). Sodium citrate (1.5 mg/g vehicle) and citric acid monohydrate (1 mg/g vehicle) were dissolved in purified water (316 mg/g vehicle) and the pH adjusted to 4.5. The buffer solution was added to the cipamfylline solution followed by heating to 70-75° C. Arlacel® 165 (20 mg/g vehicle), cetostearyl alcohol (40 mg/g vehicle) and liquid paraffin (140 mg/g vehicle) were mixed and heated to 70-75° C. The aqueous phase was slowly added to the oil phase. The resulting cream was thoroughly homogenised followed by cooling to room temperature with mild agitation. The resulting formulation was filled into 50 g tubes and stored at room temperature.

### Example 2

In Vivo Effects on Atopic Dermatitis in Humans of Topical Application of Cipamfylline

In a phase II, multi-centre, randomised, double-blind left/right study comparing cipamfylline cream 1.5 mg/g (formulation B) with the reference vehicle without cipamfylline and with Locoid cream 0.1% (hydrocortisone-17-butyrate 0.1% cream) in patients suffering from atopic dermatitis, patients were randomised to either group (1) cipamfylline cream 1.5 mg/g in reference vehicle vs vehicle or (2) cipamfylline cream 1.5 mg/g in reference vehicle vs Locoid cream. One g of each cream were applied twice daily for two weeks.

A total number of 108 patients with a clinical diagnosis of atopic dermatitis, with symmetrical lesions of atopic dermatitis on their arms with a minimum score of 6 in the total severity score (ratings of erythema, oedema/papulation, oozing/crusting, excoriations and lichenification on a scale from 0-3) were included. Excluded were patients with suspected infection in the treatment areas, patients who were receiving other treatment for atopic dermatitis or who were pregnant or planning to become pregnant in the course of the study.

At the end of the study, the total severity score had decreased by 17% on the vehicle treated side and by 38% on the cipamfylline treated side, the difference being statistically significant (p<0.001). At the end of treatment, the total severity score had decreased by 59% on the Locoid treated side and by 30% on the cipamfylline treated side, the difference being statistically significant (p<0.001).

The most frequently reported adverse effects were local adverse reactions such as burning, stinging and pruritus, most predominant on the side treated with vehicle only (25% of the patients vs 15% on the side treated with cipamfylline). Two patients withdrew from the study due to unacceptable adverse events (flare-up of eczema). The adverse reaction are thought to be the result of irritative components of the reference vehicle penetrating into the epidermis of the patients.

### Example 3

In Vivo Effects on Atopic Dermatitis in Humans of Topical Application of Cipamfylline Ointment

In a phase II, multi-centre, randomised, double-blind, left-right study comparing cipamfylline ointment (formulation A of Example 1) with cipamfylline cream (formulation B of Example 1) in patients suffering from atopic dermatitis, the patients were treated on one side with cipamfylline cream 1.5 mg/g and with cipamfylline ointment 2.5 mg/g on the other side. A maximum of 1.5-3 g of each formulation was applied twice daily for 3 weeks.

A total number of 52 patients with a clinical diagnosis of atopic dermatitis, with symmetric lesions of atopic dermatitis on their arms or legs with a minimum score of 6 in the total severity score (ratings of erythema, oedema/papulation, oozing/crusting, excoriations and lichenification on a scale from 0 to 3) were included. Excluded were patients with suspected infection on the treatment areas, who were receiving other treatment for atopic dermatitis, or who were pregnant or wished to become pregnant during the study.

At baseline, 75% of the patients had moderate and 20% had severe atopic dermatitis, according to the Investigator's Global Assessment.

At the end of treatment, the total severity score had decreased by 52.1% on the side treated with the reference cipamfylline cream and 55.8% on the side treated with the test cipamfylline ointment.

At the end of treatment, patients rated as “clear” or “almost clear” of symptoms according to the Investigator's Global Assessment were 29.4% of patients on the side treated with the reference cream and 34% of patients on the side treated with the test ointment.

Pruritus was assessed by each patient on a 10 cm visual analogue scale. A reduction in the pruritus score of 2.65 on the side treated with the reference cream and 3.35 on the side treated with the test ointment.

Treatment specific adverse drug reactions including application site disorders such as pruritus, burning sensation, skin irritation and exacerbation of atopic dermatitis were reported in 23.9% of patients on the side treated with the reference cream and in 13.2% of patients on the side treated with the test ointment.

The differences between the test and reference formulations were not statistically significant, but did, nevertheless, reflect a trend in favour of the test ointment.

1. A substantially non-aqueous pharmaceutical composition for dermal application comprising, as an active component, a compound of formula I 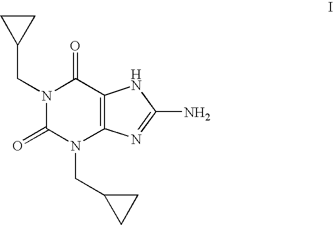
or a pharmaceutically acceptable salt thereof, and a vehicle comprising a pharmaceutically acceptable, substantially non-aqueous solvent in which the active component has a solubility of at least about 6.0 g/L at ambient temperature, said solvent being capable of effecting penetration of a therapeutically effective amount of the active component into the epidermis.

2. A composition according to claim 1, wherein the solvent is one in which the active component has a solubility of at least about 7 g/L, such as at least about 8 g/L, at least about 9 g/L, at least about 10 g/L, at least about 11 g/L, at least about 12 g/L, at least 13 g/L, at least about 14 g/L, at least about 15 g/L, at least about 16 g/L, at least about 17 g/L, or at least about 18 g/L, at ambient temperature.

3. A composition according to claim 2, wherein the solvent is selected from the group consisting of alkylene glycols or alkylene glycol ethers, e.g. propylene glycol, butylene glycol, diethylene glycol or diethylene glycol ether.

4. A composition according to any of claims 1-3, wherein the vehicle further comprises a water-in-oil emulsifier.

5. A composition according to claim 4, wherein the emulsifier is selected from the group consisting of polyoxyalkylene C₁₂₋₂₀ alkyl ether, such as polyoxyethylene-2-cetyl ether, polyoxyethylene-2-lauryl ether, polyoxyethylene-2-oleyl ether or polyoxyethylene-2-stearyl ether, sorbitan oleate, sorbitan isostearate, sorbitan sesquioleate, glycerol esters of isoostearic acid and adipic acid, polyglyceryl-3-diisostearate and polyglyceryl-6-hexaricinoleate.

6. A composition according to any of claims 1-5, wherein the vehicle comprises
(a) a solvent for the active component in an amount of about 5-20% w/w, e.g. about 10-15% w/w,
(b) a water-in-oil emulsifier in an amount of about 1.5-7.5% w/w, e.g. about 3-5% w/w, and
(c) an oil base as the remainder.

7. A composition according to claim 6, herein the amount of active substance is in the range of from about 0.5 mg/g to about 3.5 mg/g of vehicle, preferably from about 1.0 to about 3.0 mg/g of vehicle, in particular about 2.5 mg/g of vehicle.

8. Use of a pharmaceutical composition according to any of claims 1-7 for the manufacture of a medicament for the prophylaxis or treatment of dermal inflammatory diseases or conditions.

9. The use of claim 8, wherein the dermal disease or condition is dermatitis, such as atopic dermatitis, seborrheic dermatitis or contact dermatitis, urticaria, pruritis or eczema.

10. A method of preventing or treating dermal inflammatory diseases or conditions, the method comprising dermally applying to a site of inflammation, an effective amount of a pharmaceutical composition according to any of claims 1-7.

11. The method of claim 10, wherein the dermal inflammatory disease or condition is dermatitis, such as atopic dermatitis, seborrheic dermatitis or contact dermatitis, urticaria, pruritis or eczema.
